(19) United States
(12) Patent Application Publication
(10) Pub. No.: US 2010/0003275 A1
(43) Pub. Date: Jan. 7, 2010

(54) Immunostimulatory Composition comprising Lipoprotein in Microalgae Extract

(75) Inventors: David Stanley Pasco, Oxford, MS (US); Nirmal Derek Ceri Pugh, Oxford, MI (US)

Correspondence Address: HUNTON & WILLIAMS/NEW YORK;INTELLECTUAL PROPERTY DEPT., 1900 K STREET, N.W., SUITE 1200, WASHINGTON, DC, 20006-1109, US

(73) Assignee: THE UNIVERSITY OF MISSISSIPPI, University, MS (US)

(21) Appl. No.: 12/440,552

(22) PCT Filed: Sep. 10, 2007
(86) PCT No.: PCT/US2007/078008

§ 371 Date: Apr. 13, 2009

Related U.S. Application Data
(60) Provisional application No. 60/824,952, filed on Sep. 8, 2006.
(60) Provisional application No. 60/827,966, filed on Oct. 3, 2006.

(51) Int. Cl. A61K 36/02; A61P 37/04

(52) U.S. Cl. 424/195.17

(57) ABSTRACT

Potent immunostimulatory lipoproteins have been identified within the following microalgae and extracts thereof: Spirulina species, Chlorella species, Haematococcus pluvialis and Aphanizomenon flos-aquae. This lipoprotein component can be extracted from the microalgae or microalgae spent material using various procedures. The resulting preparations exhibit extremely potent immunostimulatory activity. These preparations are potentially useful as a botanical or pharmaceutical preparation to improve immune function. Methods are also disclosed for the chemical and bioactivity based standardization of immunostimulatory microalgae extracts and the raw material.

### FIELD OF THE INVENTION

The present invention relates to the identification of immunostimulatory lipoproteins within food grade microalgae and extracts thereof (Spirulina species, Chlorella species, Haematococcus pluvialis, and Aphanizomenon flos-aquae). These lipoproteins are potent activators of monocytes and they represent a significant immunostimulatory component distinct from the immunostimulatory polysaccharides previously identified in some of these extracts by these inventors. The present invention also relates to methods for the chemical and bioactivity based standardization of immunostimulatory microalgae extracts and the raw material. It also relates to methods for the treatment and/or prevention of a variety of disease conditions using the preparations of this invention.

### BACKGROUND OF THE INVENTION

During the past three decades immunotherapy has become an important approach for treating human diseases and conditions through the use of regimens designed to modulate immune responses. This is particularly important in pathological conditions where the immune system becomes compromised. Studies conducted in disease models and clinical trials demonstrate that augmenting host defense mechanisms is useful in treatment and prophylaxis against microbial infections, immunodeficiencies, cancer, and autoimmune disorders (1-5). Immune enhancing protocols may also have utility for promoting wound healing. In the process of wound healing, macrophages exhibit a principal role by modulating cellular proliferation and new tissue formation/regeneration. They also function as phagocytes, debridement agents and produce growth factors that influence the angiogenesis stage of wound repair (6).

Most immunostimulants of natural origin are high molecular weight polysaccharides, glycoproteins or complex peptides (1). For example, three fungal polysaccharides derived from Schizophyllum commune (schizophyllan), Lentinus edodes (lentinan) and Coriolus versicolor (krestin) have been clinically used in Japan as biological response modifiers (4). Another polysaccharide, acemannan (isolated from Aloe vera), is licensed by the United States Department of Agriculture for the treatment of fibrosarcoma in dogs and cats (7). There are a few small molecular weight immunostimulants derived from natural products such as the glycosphingolipid KRN-7000 (8). Several immunostimulants of synthetic origin also have been developed that include compounds like isoprinosine and muramyl peptides (2). A number of other immunomodulators of endogenous origin have been developed using recombinant technologies that have gained FDA approval. These agents include colony-stimulating factors, interferons and recombinant proteins (5). However, these compounds often have short half-lives and it is difficult to determine optimal dosage and appropriate combinations.

Although current immunostimulants show promise, there is still a need to develop more potent agents and increase the arsenal of available drugs for immunotherapy. One source of chemically diverse compounds that can be used for drug discovery of immunostimulants is natural products. For centuries natural products have been exploited as therapeutically useful agents, many of which are in clinical use today. Interest in natural products as a means to drug discovery is based on their unparalleled molecular diversity and rich spectrum of biological activities (9).

Since ancient times, microalgae have been used as a nutrient-dense food source. Historical records indicate that microalgae such as Spirulina platensis was consumed by tribes around Lake Chad in Africa and by the Aztecs living near Lake Texcoco in Mexico (10). During the last several decades there has been increasing interest in the commercial production of food-grade microalgae for human consumption and as feed for livestock. Among the various microalgae that have been explored for their commercial potential Spirulina species, Chlorella species and Aphanizomenon flos-aquae (AFA) are three major types that have been successfully produced and are in widespread use. Other food-grade microalgae include Dunaliella salina and Haematococcus pluvialis.

Both anecdotal reports and recent studies on the consumption of food-grade microalgae have reported enhanced immune function in both animals and humans. Oral administration of Chlorella vulgaris has been correlated with enhanced natural killer cell activity (11) and granulocyte-macrophage progenitor cells (12) in mice infected with Listeria monocytogenes. Dietary Spirulina platensis increases macrophage phagocytic activity in chickens (13) and Spirulina fusiformis exhibits chemopreventive effects in humans (14). Human consumption of AFA has been reported to produce changes in immune cell trafficking and enhanced immune surveillance (15). The active components for all these effects have not been conclusively established.

### Chlorella Polysaccharides and Glycoproteins

A number of polysaccharides have been identified from Chlorella species that possess biological activity. In U.S. Pat. No. 4,533,548 an acidic polysaccharide was isolated from Chlorella pyrenoidosa that exhibits antitumor and antiviral activity (16). The glycosyl composition for this polysaccharide was mostly rhamnose, with minor amounts of galactose, arabinose, glucose and glucuronic acid. Another polysaccharide, isolated from marine Chlorella minutissima, reported in U.S. Pat. No. 4,831,020, appears to have tumor growth-inhibiting effects. However, no molecular weight or glycosyl composition was reported (17).

In U.S. Pat. No. 4,786,496, the lipid fraction (glycolipid portion) of marine Chlorella species displayed antitumor properties (18). Several glycoproteins have also been isolated from Chlorella species. For example, U.S. Pat. No. 4,822,612 reported a 45,000 dalton glycoprotein that has anticancer effects (19). Various other glycoproteins (20-23) and glyceroglycolipids (24) that may have immunopotentiating and antitumor properties also have been reported in the scientific literature. None of these compounds are polysaccharides.

### Spirulina Polysaccharides

Several different types of polysaccharides that exhibit biological activity have been isolated from Spirulina species. For example, the sulfated polysaccharide calcium spirulan inhibits tumor invasion and metastasis (25). Calcium spirulan (molecular weight 74,600 daltons) is composed of rhamnose (52.3%), 3-O-methylrhamnose (32.5%), 2,3-di-O-methylrhamnose (4.4%), 3-O-methylxylose (4.8%), uronic acids (16.5%) and sulfate (26).

U.S. Pat. No. 5,585,365 discloses that an antiviral polysaccharide with a molecular weight between 250,000 and 300,000 daltons was isolated from Spirulina species using hot water extraction (27). This polysaccharide is composed of rhamnose, glucose, fructose, ribose, galactose, xylose, mannose, glucuronic acid and galacturonic acid. A number of other low molecular weight polysaccharides that range between 12,600 and 60,000 daltons recently have been isolated from Spirulina species (28-30).

### Previous Work by the Inventors

The present inventors have characterized novel polysaccharide preparations from the microalgae Spirulina platensis, Chlorella pyrenoidosa and Aphanizomenon flos-aquae (31). These are high molecular weight preparations that contain polysaccharides with methylated and acetylated sugars and therefore are extractable to some extent with water and also under more non polar conditions such as with aqueous alcohol.

In the present invention the inventors have applied the aqueous alcohol extraction method to quantitatively extract potent immunostimulatory lipoproteins from the following food-grade microalgae: Spirulina platensis, Chlorella pyrenoidosa, Aphanizomenon flos-aquae, and Haematococcus pluvialis. There has not been a report of the existence of immunostimulatory lipoproteins within these microalgae.

### Monocyte/Macrophage Activation System

One way to determine immunostimulatory activity is to use a biological assay involving macrophages. Monocytes/macrophages are found in practically every tissue of the body where they are critical in coordinating immune responses and numerous biological processes (32). They play a major role in phagocytosis, immune surveillance, wound healing, killing of microbes and tumor cells, and antigen presentation to T lymphocytes (33). In cancer, macrophages mediate tumor cytotoxicity functions through the production of cytokines and other immune factors (34). In order for macrophages to play a major role in adaptive and innate immunity they must respond effectively to environmental agents by first becoming activated (35). Macrophage activation is mediated by proinflammatory transcription factors such as nuclear factor kappa B (NF-kappa B). Such transcription factors then control and modulate the activation/repression of an array of genes that mediate a variety of immune responses.

In unstimulated macrophages, NF-kappa B exists as inactive heterodimers sequestered by inhibitory-kappa B (I-kappa B) proteins within the cytosol. Agents that cause I-kappa B proteins to dissociate and degrade allow for the translocation of NF-kappa B dimers to the nucleus where they can activate transcription of downstream genes (36). Target genes regulated by NF-kappa B include proinflammatory cytokines, chemokines, inflammatory enzymes, adhesion molecules and receptors (37).

In this invention a transcription factor based assay for NF-kappa B in human monocytes was used to guide extraction, and characterization of immunostimulatory lipoprotein preparations from food-grade microalgae.

### SUMMARY OF THE INVENTION

The inventors have identified within the commonly used food-grade microalgae, such as, Spirulina platensis, Chlorella pyrenoidosa, Haematococcus pluvialis and Aphanizomenon flos-aquae potent immunostimulatory lipoproteins. Extracts from the microalgae have been prepared that contain substantial amounts of these lipoproteins and these preparations exhibit potent immune enhancing properties. One of these properties is the activation of monocytes.

In general, the invention comprises immunostimulatory lipoproteins isolated from food-grade microalgae. According to one embodiment of the invention, immunostimulatory lipoproteins are isolated from Spirulina platensis microalgae extractable by a solvent. According to another embodiment, the immunostimulatory activity of this lipoprotein preparation is manifested by monocyte/macrophage activation. According to another embodiment, the immunostimulatory lipoproteins are extracted from the microalgae Chlorella pyrenoidosa. According to another embodiment, the immunostimulatory lipoproteins are extracted from the microalgae Aphanizomenon flos-aquae. According to another embodiment, the immunostimulatory lipoproteins are extracted from the microalgae Haematococcus pluvialis. According to another embodiment, a dietary supplement comprises any one of the previous immunostimulatory lipoprotein preparations and an acceptable carrier or excipient for dietary supplements.

According to another embodiment, a method of enhancing immune function in an individual in need of such treatment, comprises administering to said individual an effective amount of the microalgae-derived lipoprotein-containing pharmaceutical composition or dietary supplement. According to another embodiment, the individual is suffering from a viral, bacterial or fungal infection. According to another embodiment, the individual is suffering from cancer. According to another embodiment, the individual is suffering from an immune deficiency. According to another embodiment, the individual is a human being. According to another embodiment, the individual is an animal.

According to another embodiment, a method of treating an individual with an immunostimulatory lipoprotein preparation in order to provide to the individual a stimulation of monocyte/macrophage activity comprises administering to the individual an effective amount of a lipoprotein preparation extracted from food-grade microalgae in combination with an acceptable carrier. According to another embodiment, the immunostimulatory lipoprotein preparation is administered to enhance wound healing. According to another embodiment, the immunostimulatory lipoprotein preparation is administered to treat cancer. According to another embodiment, the immunostimulatory lipoprotein preparation is administered to treat immunodeficiency. According to another embodiment, the immunostimulatory lipoprotein preparation is administered to treat a viral, bacterial or fungal infection. According to another embodiment, the individual is a human being. According to another embodiment, the individual is an animal. According to another embodiment, a method of treating an individual with an immunostimulatory lipoprotein preparation in order to provide to the individual a stimulation of monocyte/macrophage activity comprises administering to the individual an effective amount of a lipoprotein preparation extracted from Spirulina platensis in combination with an acceptable carrier. According to another embodiment, a method of treating an individual with an immunostimulatory lipoprotein preparation in order to provide to the individual a stimulation of monocyte/macrophage activity comprises administering to the individual an effective amount of a lipoprotein preparation extracted from Chlorella pyrenoidosa. According to another embodiment, a method of treating an individual with an immunostimulatory lipoprotein preparation in order to provide to the individual a stimulation of monocyte/macrophage activity comprises administering to the individual an effective amount of a lipoprotein preparation extracted from Aphanizomenon flos-aquae. According to another embodiment, a method of treating an individual with an immunostimulatory lipoprotein preparation in order to provide to the individual a stimulation of monocyte/macrophage activity comprises administering to the individual an effective amount of a lipoprotein preparation extracted from Haematococcus pluvialis.

### BRIEF DESCRIPTION OF THE FIGURES

FIG. 1. Proteinase K digestion and SDS polyacrylamide gel analysis of Spirulina platensis lipoprotein preparation.

FIG. 2. Lipoprotein lipase digestion of Spirulina platensis lipoprotein preparation.

FIG. 3. Proteinase K digestion and SDS polyacrylamide gel analysis of Aphanizomenon flos-aquae lipoprotein preparation.

FIG. 4. Lipoprotein lipase digestion of Aphanizomenon flos-aquae lipoprotein preparation.

FIG. 5. Proteinase K digestion and SDS polyacrylamide gel analysis of Haematococcus pluvialis lipoprotein preparation.

FIG. 6. Lipoprotein lipase digestion of Haematococcus pluvialis lipoprotein preparation.

FIG. 7. Proteinase K digestion and SDS polyacrylamide gel analysis of Chlorella pyrenoidosa lipoprotein preparation.

FIG. 8. Lipoprotein lipase digestion of Chlorella pyrenoidosa lipoprotein preparation.

### DETAILED DESCRIPTION OF THE INVENTION

This invention describes the identification of immunostimulatory lipoproteins within the following microalgae: Spirulina platensis, Chlorella pyrenoidosa, Aphanizomenon flos-aquae and Haematococcus pluvialis. These lipoproteins are potent activators of monocytes and represent a significant immunostimulatory component within these microalgae. The lipoproteins that have been identified in these microalgae contain a specific structural moiety that make them potent immunostimulants. Based on previous research it is currently believed that this structural moiety is unique to prokaryotic organisms (39).

It is of commercial interest to produce dietary supplement extracts from these microalgae that concentrate the immune enhancing components. One major immune enhancing component is the lipoproteins described in the current patent. The identified lipoproteins are however difficult to extract due to their amphipathic nature: they contain both a polar component (protein) and a non-polar component (lipid). For example, very low amounts of lipoprotein are extracted from these microalgae using solvents such as hot water, 100% alcohol and organic solvents.

In the present invention two solvent systems are described that are capable of quantitatively extracting the immunostimulatory lipoproteins. Both solvent systems can be used commercially to produce extracts that concentrate the immunostimulatory lipoproteins. The first solvent system uses aqueous alcohol at elevated temperatures (e.g. 50% ethanol at 80° C.). The extracts produced by this aqueous alcohol extraction system were previously described in an earlier patent by the present inventors (31). In this earlier patent, the aqueous alcohol extraction procedure was developed to preferentially extract the immunostimulatory polysaccharides. In the present invention it was discovered that these extracts also contain high amounts of immunostimulatory lipoproteins, in addition to the polysaccharides.

The second solvent system described in this patent uses detergents to produce extracts that concentrate the amount of immunostimulatory lipoproteins. Crude extracts can be obtained by extraction of the microalgae raw material using a detergent, a surfactant, an emulsifier or any combination thereof. Useful surfactants or detergents include food-grade surfactants or detergents, i.e. surfactants or detergents suitable for mammal or human consumption, such as e.g. Saponins obtained from sources such as Quillaja saponaria or Yucca schidigera.

Both aqueous alcohol and detergent solvents can also be used to produce extracts from microalgae spent (waste) material. Microalgae spent material is produced when the microalgae is extracted with solvents (e.g. water or non-polar solvents) to obtain other important substances. This spent material is viewed by the industry as relatively useless or only used as filler or animal feed. There is currently no use for this spent material in the dietary supplement industry. However, in the present invention, microalgae spent material is viewed as having value since it will contain varying amounts of lipoproteins. This spent material could therefore be used as a dietary supplement for enhancing immune function or it could be further extracted to produce concentrated immunostimulatory extracts. For example, Spirulina or Aphanizomenon flos-aquae can be extracted with water to obtain phycocyanin (and/or water extractable polysaccharides). The resulting spent material would still contain substantial amounts of immunostimulatory lipoproteins that could be recovered by extraction with either aqueous alcohol or detergent solvents. A second example is Haematococcus which is of commercial interest as a rich source of astaxanthin. Since extraction of astaxanthin involves the use of non-polar solvents, the spent material would still contain substantial amounts of immunostimulatory lipoproteins that could be recovered by extraction with either aqueous alcohol or detergent solvents.

The present invention also discloses two methods that can be used for product standardization. Both methods can be used for standardizing either extract material or the raw material. The purpose of standardization is to ensure that each batch of product material contains the same level of active component(s).

The first standardization method is preparing a bioactivity standardized microalgae product containing an effective amount of immunostimulatory activity. In this method, microalgae product material is tested in vitro for activation of immune cells and the bioactivity is then compared to a standard preparation immunostimulatory value to determine a standardized activity value of the product. Bioactivity based standardization of product material is important when the chemical content of the active components do not correlate with biological activity due to unknown structure-activity relationships and/or complex interactions between multiple actives. Under such circumstances the amount of active substances is not sufficient to reflect the potency of the product material and standardization through the use of a biological assay is more relevant and appropriate. This approach of bioactivity standardization has been used by the pharmaceutical industry for biologics such as insulin and cytokines.

The second standardization method is preparing a chemically standardized microalgae product containing an effective amount of immunostimulatory lipoproteins. The chemical marker used for standardization is 2,3-dihydroxypropyl cysteine. This modified cysteine amino acid is thought to be unique to lipoproteins that are immunostimulatory from prokaryotic organisms (39). In this method, microalgae product material is tested for the amount of 2,3-dihydroxypropyl cysteine and then compared to the amount of 2,3-dihydroxypropyl cysteine in a standard preparation to determine a standardized value of the product.

### Methods

### Monocyte Assay

The transcription factor-based bioassay for activation of NF-kappa B in THP-1 human monocytes/macrophages was used to evaluate the immunostimulatory potential of lipoproteins extracted from the microalgae. This assay measures immunostimulatory activity as indicated by increased expression of a NF-kappa B-driven luciferase reporter. THP-1 human monocytes (American Type Culture Collection, Rockville, Md.) were cultured in RPMI 1640 medium supplemented with fetal bovine serum (10% v/v) and amikacin (60 mg/L) at 37° C., under 5% CO₂ and 95% air. Actively growing cells were transiently transfected using DEAE-dextran (10 μg/1×10⁶ cells) and the pBIIXLUC reporter plasmid (1 μg/1×10⁶ cells). This plasmid, a gift from Dr. Riccardo Dalla-Favera, contains two copies of NF-kappa B motif from HIV/IgK (38). Transfection solution containing THP-1 cells was incubated for 7 minutes in a 37° C. water bath. The transfected cells were then resuspended in 10% FBS, RPMI 1640 medium and plated out in 96-well plates at a cell density of 2×10⁵ cells per well. After 24-hours, test samples were added to transfected cells. Cells were harvested and luciferase activity measured four hours after addition of samples. Cells were harvested using 96-well filter plates and lysed using 40 μL of luciferase mix (1:1, luciferase assay reagent: 1×PBS, 1 mM Ca and Mg). Luciferase assay kit was purchased from Promega (Madison, Wis.). Light emission was measured using a Packard microplate scintillation counter in single photon mode. Activation is reported as a percentage relative to maximal activation of NF-kappa B by 10 μg/mL LPS (E. coli, serotype 026:B6, Sigma Chemical Co., St. Louis, Mo.) which was used as a positive control.

This monocyte assay is an example of an in vitro test system that can be used for bioactivity based standardization of microalgae extracts and product material.

First Solvent System: Extraction of Active Lipoproteins from Microalgae Using Aqueous Alcohol

For each microalgae, 330 g of dry raw material was extracted twice at 70° C. with water, first with 3.6 L for 45 minutes and then with 3.0 L for 45 minutes. Water extracts were discarded since they contained minimal immunostimulatory activity when tested in the monocyte assay (data not shown). The marc material left over after the water extraction was freeze-dried and re-extracted twice at 90° C. with 50% ethanol in sealed containers, first with 1.8-2.4 L for 45 minutes and then with 0.9-1.2 L for 45 minutes. Supernatants from both extractions were combined following centrifugation. The ethanol concentration of the supernatant was adjusted to 72.5% by the addition of one volume of cold 95% ethanol. Following incubation at −20° C. overnight, precipitates were collected by centrifugation and subsequently washed with cold 95% ethanol. The isolated material was dried and represented a crude extract containing immunostimulatory lipoproteins. Alternatively, microalgae may be extracted twice at 90° C. with 50% ethanol in sealed containers without the prior water extraction and lipoprotein yields and activity are similar to preparations where microalgae were first extracted with water.

The extracts produced using the aqueous alcohol extraction system at elevated temperatures exhibit potent activation of monocytes and represents product material of commercial interest that is suitable for consumption by a subject. These extracts contain concentrated levels of the immunostimulatory lipoproteins that are present within the microalgae described in this invention. This solvent system can be used to create extracts from raw material or spent material for the following microalgae described in this invention: Spirulina species, Chlorella species, Aphanizomenon flos-aquae and Haematococcus pluvialis.

Second Solvent System: Extraction of Active Lipoproteins from Microalgae Using Detergents

Refer to EXAMPLE 6 and EXAMPLE 7.

### Lipoprotein Characterization and Identification

Lipoprotein lipase treatment: aqueous alcohol extracts from each microalgae were dissolved in 1% n-octyl-β-D-glucopyranoside (octylglucoside). Octylglucoside insoluble material (inactive in monocyte assay, data not shown) was removed by centrifugation and discarded. To determine sensitivity to lipoprotein lipase, samples were adjusted to a final concentration of 0.5% octylglucoside, 10 μM AEBSF protease inhibitor cocktail solution (Sigma) and 0.2% BSA (Sigma, No. A-9418). Samples were incubated at 37° C. for 16 hours with 39,600 units/ml (1 mg/ml) of lipoprotein lipase from Pseudomonas species (Sigma No. L9656). Control samples (without lipoprotein lipase) were run under identical conditions. Activity of lipoprotein lipase treated and untreated samples were evaluated using the monocyte assay.

Proteinase K treatment and SDS polyacrylamide gel analysis: aqueous alcohol extracts from each microalgae were dissolved in 1% octylglucoside at 10 mg/ml. Octylglucoside insoluble material (inactive in monocyte assay, data not shown) was removed by centrifugation and discarded. Samples were incubated with 0.1 mg/ml (3.6 units/ml) proteinase K from Tritirachium album (Sigma) in 50 mM TRIS (pH 8.5), 5 mM B-mercaptoethanol, and 5 mM CaCl₂ for 2 hours at 50° C. Digests were then heated at 98° C. for 10 minutes. Control samples (without proteinase K) were run under identical conditions. Activity of proteinase K treated and untreated samples were evaluated using the monocyte assay.

For SDS polyacrylamide gel analysis, 100 μg of each sample (proteinase K treated and untreated) was mixed with 1 volume of Tris-Tricine sample buffer (Bio-Rad) and loaded in nonadjacent lanes of a 16.5% Tris-Tricine precast gel (Ready Gel, Bio-Rad). Wide molecular weight range and ultra-low molecular weight range protein markers (Sigma) were run in each gel. Individual gel lanes were cut into 12 equal sections (0.5 cm/section), each section was crushed and then extracted with 200 μl of 1% octylglucoside, 50 mM TRIS (pH 8.5), 5 mM CaCl₂ at 95° C. for 5 minutes. Supernatants of sample were collected and evaluated for activity in the monocyte assay.

2,3-dihydroxypropyl cysteine composition analysis: the following procedure was developed based on modifying a published method (40). Aqueous alcohol extracts from each microalgae were completely dissolved in 4% sodium dodecyl sulfate (SDS), 10 mM TRIS at a concentration of 20 mg/ml. Dissolved samples were incubated with 1.5 mM β-mercaptoethanol at 98° C. for 10 minutes. After cooling to room temperature, samples were diluted 40 times with distilled water to reduce SDS concentration to 0.1%. Low molecular weight substances and SDS were removed by subjecting the diluted samples to a 5,000 MWCO ultrafiltration device from Millipore. Samples were then incubated with 0.1 mg/ml (3.9 units/ml) proteinase K from Tritirachium album (Sigma) in 50 mM TRIS (pH 8.5) and 5 mM CaCl₂ for 2 hours at 50° C. The purpose of proteinase K treatment was to digest the majority of the protein away from the lipopeptide moiety of the lipoproteins. After proteinase K digestion, samples were solvent partitioned against an equal volume of phenol. The phenol layer was then partitioned 3 times against equal volumes of water. The final phenol layer (containing the lipopeptide moiety of the lipoproteins) was then freeze-dried.

Freeze-dried samples were sent to Texas A&M University, Protein Chemistry Laboratory for analysis of 2,3-dihydroxypropyl cysteine using the following protocol. Samples were hydrolyzed using 4N methanesulfonic acid for 18 hours at 102° C. Hydrolysates were analyzed using a Hewlett Packard AminoQuant System. In this system the hydrolyzed amino acids undergo precolumn derivitization with o-phthalaldehyde and are then separated by reverse phase HPLC and detected using fluorescence. Quantitation of 2,3-dihydroxypropyl cysteine was achieved by using N-palmitoyl-S-[2,3-bis(palmitoyloxy)-propyl]-(2RS)-propyl]-[R]-cysteinyl-[S]-seryl-[S]-lysyl-[S]-lysyl-[S]-lysyl-[S]-lysine (Pam₃CSK₄, purchased from InvivoGen, San Diego, Calif.) as a standard. Pam₃CSK₄ is a synthetic tripalmitoylated bacterial lipopeptide analogue that, after hydrolysis with methanesulfonic acid, contains a known amount 2,3-dihydroxypropyl cysteine.

The modified cysteine amino acid, 2,3-dihydroxypropyl cysteine, represents a chemical marker that can be used for preparing chemically standardized microalgae extracts and product material containing an effective amount of immunostimulatory lipoproteins. The use of 2,3-dihydroxypropyl cysteine as a marker to standardize extracts from these microalgae is not known in the art.

### Example 1

### Identification of Immunostimulatory Lipoproteins from Spirulina platensis

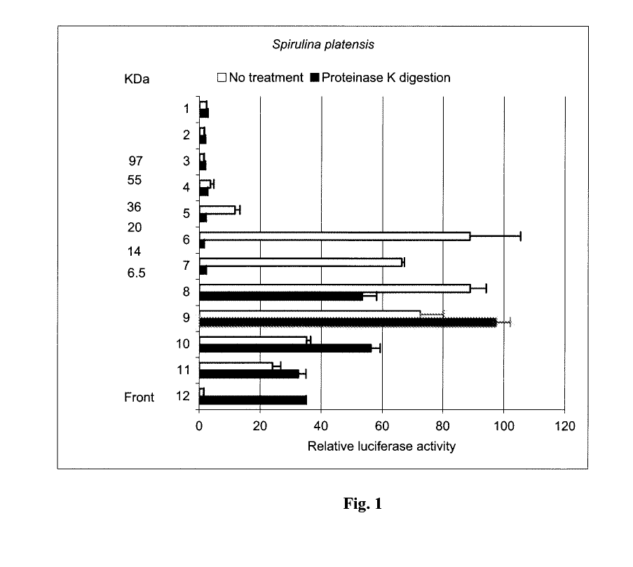

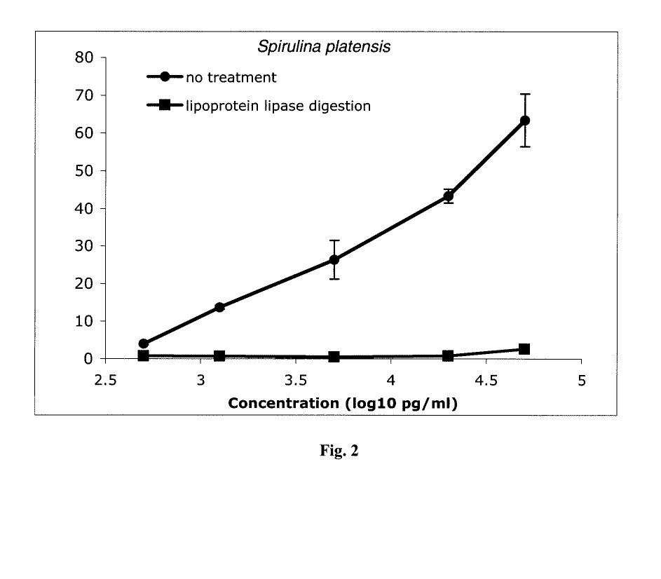

Using the aqueous alcohol extraction procedure an extract was prepared from Spirulina platensis. This extract is 3.1% of the dry weight of the microalgae raw material and is a potent activator of monocytes as determined by the monocyte assay and represents material suitable for consumption by a subject. This material was treated with proteinase K to determine if protein was responsible for the activity detected in the monocyte assay. No difference in luciferase activity was seen between untreated and proteinase K treated material indicating that proteins were not directly responsible for activation of the monocytes (data not shown). However, FIG. 1 shows that although protein is not directly responsible for the activation of the monocytes, protein is part of the molecule that is responsible for this activity. This is indicated by the substantial reduction in the apparent size of the active compounds following proteinase K treatment as determined by fractionation on an SDS-polyacrylamide gel. While the bulk of the activity in the untreated sample fractionated between 20 and <6.5 kDa, proteinase K treated material fractionated between 6.5 kDa and the gel front. This result is similar to those obtained with bacterial lipoproteins in that proteinase K digestion does not reduce the activity of the lipoproteins (i.e. the protein component of the lipoprotein is not necessary for monocyte activation) but proteinase K treatment does reduce the size of the lipoproteins when fractionated on an SDS-polyacrylamide gel (41). The results presented in FIG. 2 show that the activity present in the Spirulina platensis extract is completely abrogated by treatment with lipoprotein lipase. This result together with the results presented in FIG. 1 confirms that the activity in this extract is due to lipoproteins. This approach has been used to verify that the lipoprotein fraction from S. aureus is responsible for monocyte activation (41).

### Example 2

### Identification of Immunostimulatory Lipoproteins from Aphanizomenon flos-aquae

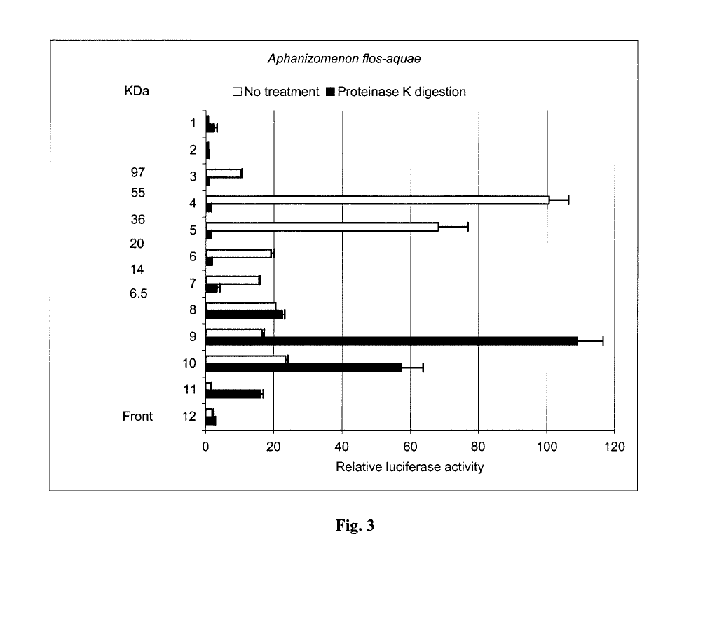

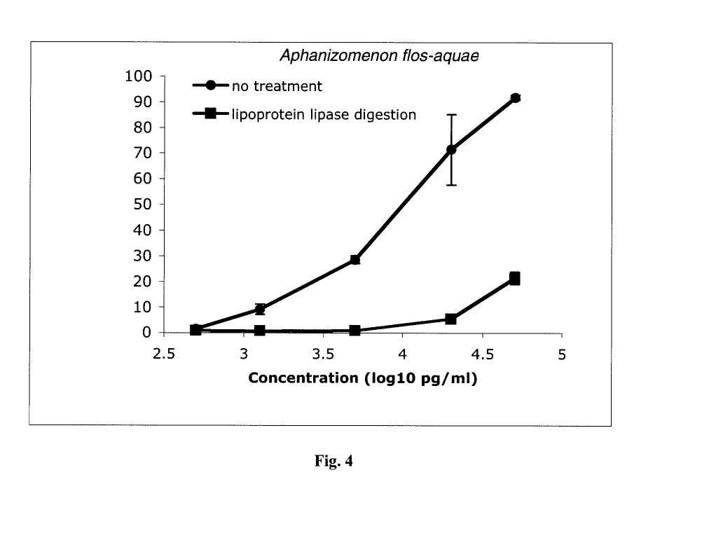

Using the aqueous alcohol extraction procedure an extract was prepared from Aphanizomenon flos-aquae. This extract is 2.3% of the dry weight of the microalgae raw material and is a potent activator of monocytes as determined by the monocyte assay and represents material suitable for consumption by a subject. This material was treated with proteinase K to determine if protein was responsible for the activity detected in the monocyte assay. No difference in luciferase activity was seen between untreated and proteinase K treated material indicating that proteins were not directly responsible for activation of the monocytes (data not shown). However, FIG. 3 shows that although protein is not directly responsible for the activation of the monocytes, protein is part of the molecule that is responsible for this activity. This is indicated by the substantial reduction in the apparent size of the active compounds following proteinase K treatment as determined by fractionation on an SDS-polyacrylamide gel. While the bulk of the activity in the untreated sample fractionated between 55 and 6.5 kDa, proteinase K treated material fractionated between 6.5 kDa and the gel front. This result is similar to those obtained with bacterial lipoproteins in that proteinase K digestion does not reduce the activity of the lipoproteins (i.e. the protein component of the lipoprotein is not necessary for monocyte activation) but proteinase K treatment does reduce the size of the lipoproteins when fractionated on an SDS-polyacrylamide gel (41). The results presented in FIG. 4 show that the activity present in the Aphanizomenon flos-aquae extract is completely abrogated by treatment with lipoprotein lipase. This result together with the results presented in FIG. 3 confirms that the activity in this extract is due to lipoproteins. This approach has been used to verify that the lipoprotein fraction from S. aureus is responsible for monocyte activation (41).

### Example 3

### Identification of Immunostimulatory Lipoproteins from Haematococcus pluvialis

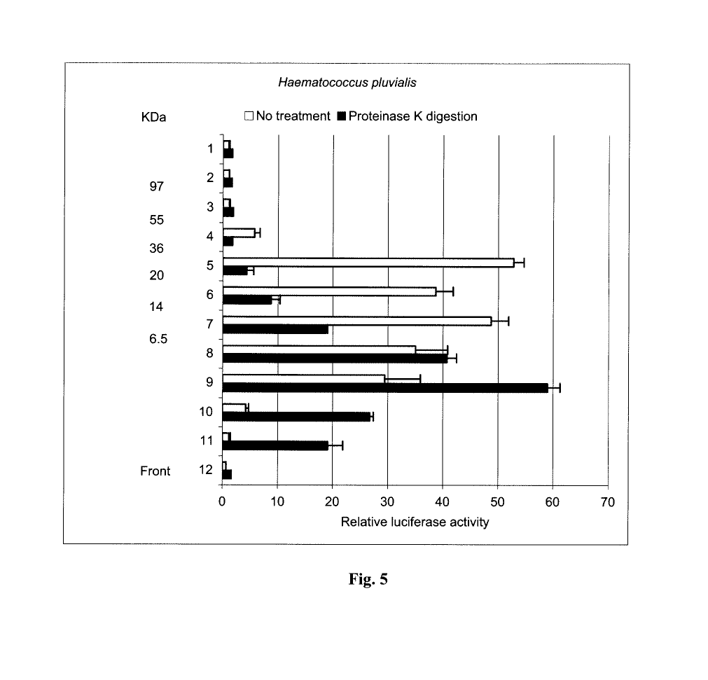

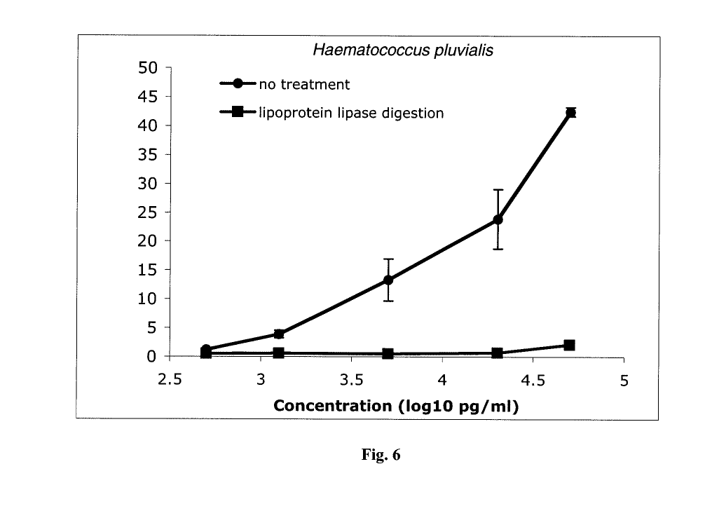

Cultivation of food-grade Haematococcus pluvialis is of commercial interest as a rich source of astaxanthin. Since extraction of astaxanthin involves the use of non-polar solvents, the spent (or waste) material left over after extraction may contain useful polar substances such as polysaccharides and lipoproteins. To investigate this possibility, the aqueous alcohol extraction procedure was used to prepare an extract from commercial dried Haematococcus pluvialis spent material. This extract is 1.8% of the dry weight of the original microalgae spent material and is a potent activator of monocytes as determined by the monocyte assay and represents material suitable for consumption by a subject. This material was treated with proteinase K to determine if protein was responsible for the activity detected in the monocyte assay. No difference in luciferase activity was seen between untreated and proteinase K treated material indicating that proteins were not directly responsible for activation of the monocytes (data not shown). However, FIG. 5 shows that although protein is not directly responsible for the activation of the monocytes, protein is part of the molecule that is responsible for this activity. This is indicated by the substantial reduction in the apparent size of the active compounds following proteinase K treatment as determined by fractionation on an SDS-polyacrylamide gel. While the bulk of the activity in the untreated sample fractionated between 36 and <6.5 kDa, proteinase K treated material fractionated between 6.5 kDa and the gel front. This result is similar to those obtained with bacterial lipoproteins in that proteinase K digestion does not reduce the activity of the lipoproteins (i.e. the protein component of the lipoprotein is not necessary for monocyte activation) but proteinase K treatment does reduce the size of the lipoproteins when fractionated on an SDS-polyacrylamide gel (41). The results presented in FIG. 6 show that the activity present in the Haematococcus pluvialis extract is completely abrogated by treatment with lipoprotein lipase. This result together with the results presented in FIG. 5 confirms that the activity in this extract is due to lipoproteins. This approach has been used to verify that the lipoprotein fraction from S. aureus is responsible for monocyte activation (41).

Haematococcus pluvialis spent material is viewed by the industry as relatively useless or only used as filler or animal feed. There is currently no use for this spent material in the dietary supplement industry. However, based on the above results, this spent material contains a substantial amount of immunostimulatory lipoproteins that are of commercial interest. This spent material could therefore be used as a dietary supplement for enhancing immune function or it could be further extracted to produce concentrated immunostimulatory extracts.

### Example 4

### Identification of Immunostimulatory Lipoproteins from Chlorella Pyrenoidosa

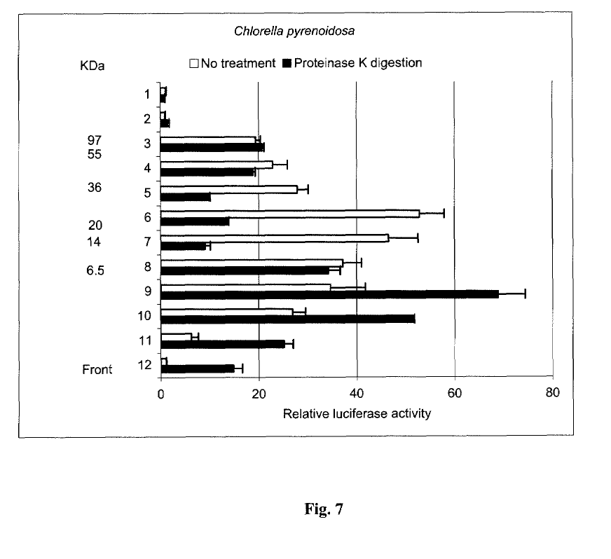

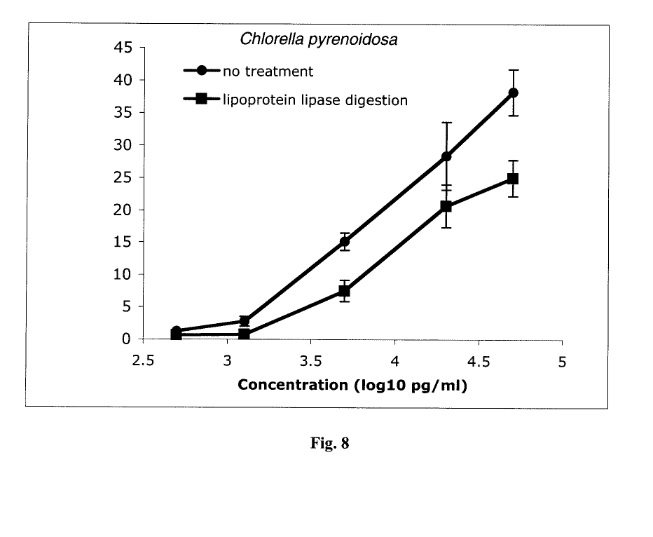

Using the aqueous alcohol extraction procedure an extract was prepared from Chlorella pyrenoidosa. This extract is 1.3% of the dry weight of the microalgae raw material and is a potent activator of monocytes as determined by the monocyte assay and represents material suitable for consumption by a subject. This material was treated with proteinase K to determine if protein was responsible for the activity detected in the monocyte assay. No difference in luciferase activity was seen between untreated and proteinase K treated material indicating that proteins were not directly responsible for activation of the monocytes (data not shown). However, FIG. 7 shows that although protein is not directly responsible for the activation of the monocytes, protein is part of the molecule that is responsible for a major portion of the activity. This is indicated by the substantial reduction in the apparent size of a major fraction of the active compounds following proteinase K treatment as determined by fractionation on an SDS-polyacrylamide gel. While the bulk of the activity in the untreated sample fractionated between 97 and <6.5 kDa, a major portion of the proteinase K treated material fractionated between 6.5 and the gel front. This result is similar to those obtained with bacterial lipoproteins in that proteinase K digestion does not reduce the activity of the lipoproteins (i.e. the protein component of the lipoprotein is not necessary for monocyte activation) but proteinase K treatment does reduce the size of the lipoproteins when fractionated on an SDS-polyacrylamide gel (41). In contrast to the results presented in the previous three examples, there is a region of activity resistant to proteinase K from 97 to 55 kDa suggesting an additional type of non-protein containing active agent. The results presented in FIG. 8 show that approximately 50% of the activity present in the Chlorella pyrenoidosa extract is abrogated by treatment with lipoprotein lipase. This result together with the results presented in FIG. 7 confirms that a major portion of the activity in this extract is due to lipoproteins. This approach has been used to verify that the lipoprotein fraction from S. aureus is responsible for monocyte activation (41).

### Example 5

### Identification of 2,3-Dihydroxyproply Cysteine in Aqueous Alcohol Extracts from Spirulina platensis, Chlorella pyrenoidosa, Aphanizomenon flos-aquae and Haematococcus pluvialis

Bacterial lipoproteins have a specific structural moiety that make them potent activators of monocytes/macrophages. The protein component of the lipoprotein is not necessary for monocyte/macrophage activation. Within the lipopeptide moiety the number and type of fatty acids may differ between lipoproteins as well as the amino acid composition. There is however a structural unit of the lipopeptide moiety that appears to be conserved in immunostimulatory bacterial lipoproteins (39). This structural unit is the modified cysteine amino acid, which after acid hydrolysis of the lipopeptide, can be detected as 2,3-dihydroxypropyl cysteine. Therefore, the identification of 2,3-dihydroxypropyl cysteine within the acid hydrolysate of an extract or fraction is strong chemical evidence for the presence of these immunostimulatory lipoproteins.

Using the aqueous alcohol extraction procedure an extract was prepared from each of the 4 microalgae described in this invention. These extracts were then analyzed for the presence of 2,3-dihydroxypropyl cysteine according to the protocols described in the Methods section. In the extracts from all 4 microalgae the 2,3-dihydroxypropyl cysteine was detected (see data below). This provides chemical evidence that these extracts contain immunostimulatory lipoproteins having the unique modified cysteine structural moiety.

The following summarizes the amount of 2,3-dihydroxypropyl cysteine (nmoles) that was detected in the aqueous alcohol extract from each microalgae:

| | 2,3-dihydroxypropyl cysteine (nmoles)/mg |
|---|---|
| Microalgae | aqueous alcohol extract |
| Aphanizomenon flos-aquae | 3.02 nmoles/mg |
| Chlorella pyrenoidosa | 12.84 nmoles/mg |
| Haematococcus pluvialis | 12.99 nmoles/mg |
| Spirulina platensis | 12.70 nmoles/mg |

### Example 6

### Preparation of Extracts Containing Immunostimulatory Lipoproteins from Microalgae Raw Material Using a Detergent Solvent System

In an earlier patent (31), the present inventors described microalgae extracts produced by extraction of raw material with aqueous alcohol at elevated temperatures. In this earlier patent, the aqueous alcohol extraction procedure was developed to preferentially extract the immunostimulatory polysaccharides. In the present invention (Examples 1-5) it was discovered that these extracts also contain high amounts of immunostimulatory lipoproteins, in addition to the polysaccharides.

In this Example, the inventors describe an alternative extraction procedure that was developed using a detergent solvent system to produce extracts that concentrate the amount of immunostimulatory lipoproteins. Crude extracts can be obtained by extraction of microalgae raw material using a detergent, a surfactant, an emulsifier or any combination therefore. Food-grade detergents (e.g. saponins from Quillaja saponaria or Yucca schidigera) are preferred since these extracts are for consumption by a subject. One advantage of using this detergent solvent system, as compared with using aqueous alcohol, is that no alcohol is used in the extraction process (which translated into a potentially lower production cost). Detergent solvents can be used to produce extracts from any one of the following microalgae: Spirulina platensis, Chlorella pyrenoidosa, Aphanizomenon flos-aquae or Haematococcus pluvialis. The use of detergent solvents to make extracts from these microalgae is not known in the art.

The following provides an example of extracting Spirulina raw material with two different detergent solvents to produce extracts that contain immunostimulatory lipoproteins. For each extraction condition 0.5 g of Spirulina platensis was extracted once using the following conditions:
Tube 1: added 6 mls of 1% octylglucoside (in water) and extracted at 37° C. for 1 hour
Tube 2: added 6 mls of 1% octylglucoside (in water) and extracted at 80° C. for 1 hour
Tube 3: added 6 mls of 1% saponin solution and extracted at 37° C. for 1 hour
Tube 4: added 6 mls of 1% saponin solution and extracted at 80° C. for 1 hour
Note: “saponin solution” refers to a solvent prepared by dissolving a crude preparation of sapogenin glycosides from Quillaja bark obtained from Sigma (cat. no. S7900) in distilled water. The percentage of the solution indicates the actual level of sapogenin glycosides in the final solvent used for extraction. Since the content of sapogenin glycosides in the Sigma product is approximately 10%, a 10% crude solution is prepared in order to obtain a 1% solution of saponins.
   Supernatant extracts were collected by centrifugation of tubes at 3000 RPM for 15 minutes. Liquid extracts were then tested directly in the monocyte assay along with an aqueous alcohol extract for comparison. The aqueous alcohol extract was prepared by extracting Spirulina platensis raw material with 50% ethanol at 80° C., without prior water extraction, according to the procedure outlined in the Methods section.

The aqueous alcohol extract was tested in the monocyte assay at 100 ng/ml and 25 ng/ml. The liquid extracts from the detergent solvent extractions were tested at concentrations equivalent 100 ng/ml and 25 ng/ml. The immunostimulatory activity of each extract tested in the monocyte assay was as follows:

| Extract | 100 ng/ml | 25 ng/ml |
|---|---|---|
| Aqueous alcohol extract = | 34.2% | 16.0% |
| 1% octylglucoside, 37° C. extract = | 27.2% | 15.0% |
| 1% octylglucoside, 80° C. extract = | 21.3% | 3.5% |
| 1% saponin solution, 37° C. extract = | 20.8% | 11.2% |
| 1% saponin solution, 80° C. extract = | 18.0% | 7.6% |

Note:
monocyte activation is expressed as a percentage relative to maximal activation of NF-kappa B by 10 μg/ml LPS.

The following conclusion is obtained from these results. Detergent solvents can be used to obtain extracts from microalgae raw material with similar immunostimulatory activity as compared with extracts obtained using aqueous alcohol as an extraction solvent. The immunostimulatory activity in these extracts indicates that lipoproteins are being extracted using the detergent solvents. For dry product material, the liquid extracts can be dried using freeze-drying, spray drying or other techniques known in the art.

### Example 7

### Preparation of Extracts Containing Immunostimulatory Lipoproteins from Microalgae Spent (Waste) Material Using a Detergent Solvent System

EXAMPLE 6 describes how detergent solvents can be used to produce immunostimulatory extracts for microalgae raw material. The purpose of this example is to demonstrate that detergent solvents can also be used to produce immunostimulatory extracts from microalgae spent material. The following provides an example using Spirulina platensis.

Four different extraction conditions were evaluated. For each extraction condition 0.5 g of Spirulina platensis was initially extracted with 6 mls of distilled water at 80° C. for 1 hour. Water extracts were discarded. The water extracts contain minimal immunostimulatory activity when tested in the monocyte assay (data not shown), but would contain other components of commercial interest such as phycocyanin and water extractable polysaccharides. The wet spent material leftover after the water extraction was re-extracted using 4 mls of detergent solvent at 37° C. for 1 hour. The following specifies the detergent solvent used for each extraction condition.

| | | | |
|---|---|---|---|
| Tube 1: | 1% saponin solution | Tube 2: | 0.5% saponin solution |
| Tube 3: | 0.25% saponin solution | Tube 4: | 0.1% saponin solution |

Note:
“saponin solution” refers to a solvent prepared by dissolving a crude preparation of sapogenin glycosides from Quillaja bark obtained from Sigma (cat. no. S7900) in distilled water. The percentage of each solution indicates the actual level of sapogenin glycosides in the final solvent used for extraction. For example, since the content of sapogenin glycosides in the Sigma product is approximately 10%, a 10% crude solution is prepared in order to obtain a 1% solution of saponins.

Supernatant extracts were collected by centrifugation of tubes at 3000 RPM for 15 minutes. Liquid extracts were then tested directly in the monocyte assay along with an aqueous alcohol extract for comparison. The aqueous alcohol extract was prepared by extracting Spirulina platensis raw material with 50% ethanol at 80° C., without prior water extraction, according to the procedure outlined in the Methods section.

The aqueous alcohol extract was tested in the monocyte assay at 100 ng/ml and 25 ng/ml. The liquid extracts from the detergent solvent extractions were tested at concentrations equivalent 100 ng/ml and 25 ng/ml. The immunostimulatory activity of each extract tested in the monocyte assay was as follows:

| Extract | 100 ng/ml | 25 ng/ml |
|---|---|---|
| Aqueous alcohol extract = | 53.8% | 25.2% |
| 1% saponin extract = | 38.0% | 19.8% |
| 0.5% saponin extract = | 28.7% | 18.8% |
| 0.25% saponin extract = | 20.8% | 6.8% |
| 0.1% saponin extract = | 28.9% | 9.6% |

Note:
monocyte activation is expressed as a percentage relative to maximal activation of NF-kappa B by 10 μg/ml LPS.

The following conclusions are obtained from these results:
1. Solvents containing a sufficient concentration of detergent can be used to obtain extracts from microalgae spent material with similar immunostimulatory activity as compared with extracts obtained using aqueous alcohol as an extraction solvent. The immunostimulatory activity in these extracts indicates that lipoprotein are being extracted using the detergent solvents. For dry product material, the liquid extracts can be dried using freeze-drying, spray drying or other techniques known in the art.
2. Extracts exhibiting the highest level of immunostimulatory activity are obtained when the extraction solvent contains a sufficient concentration of detergent. For example, the results above demonstrate that a solvent containing at least 0.5-1.0% sapogenin glycosides from Quillaja bark is necessary to produce extracts with a high level of immunostimulatory activity.

### Pharmaceutical Formulations

Since the present lipoprotein preparations maybe useful as agents for immunotherapy in the treatment of immunodeficiency disorders, cancer, wound healing and infectious diseases, the present invention includes pharmaceutical compositions containing the instant lipoprotein preparations optionally in combination with acceptable pharmaceutical carriers or excipients.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

The amount of composition administered will be dependent upon the condition being treated, the subject being treated, on the subject's weight, the severity of the affliction, the manner of administration and the judgment of the personalizing physician.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries which facilitate processing of the compositions compounds into preparation which can be used pharmaceutically. Proper formulation is dependent upon the route of administration chosen.

For injection, the agents of the invention may be formulated in aqueous solutions, preferably in physiologically compatible buffers such as Hanks solution, Ringer's solution, or physiological saline buffer. For transmucosal administration, penetrants appropriate to the barrier to be permeated are used in the formulation. Such penetrants are generally known in the art.

For oral administration, the compositions can be formulated readily by combining the active compositions with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP).

If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as fit, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a power mix of the compound and a suitable powder base such as lactose or starch.

The compositions may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active composition may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compositions may also be formulated in rectal compositions such as suppositories or retention enemas, e.g., containing conventional suppository bases such as cocoa butter or other glycerides.

In addition to the formulations described previously, the compositions may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable routes of administration may, for example, include oral, rectal, transmucosal, transdermal, or intestinal administration, parenteral delivery, including intramuscular, subcutaneous, intramedullary injections, as well as intrathecal, direct intraventricular, intravenous, intraperitoneal, intranasal, or intraocular injections.

Alternatively, one may administer the composition in a local rather than systemic manner, for example, via injection of the compound directly into an affected area, often in a depot or sustained release formulation.

Furthermore, one may administer the drug in a targeted drug delivery system, for example, in a liposome coated with an antibody specific for affected cells. The liposomes will be targeted to and taken up selectively by the cells.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a composition of the invention formulated in a compatible pharmaceutical carrier may also be prepared, placed in an appropriate container, and labeled for treatment of an indicated condition. Suitable conditions indicated on the label may include treatment of a disease.

### Dietary Supplements

Dietary supplements suitable for use in the present invention include compositions wherein the active ingredients are contained in an effective amount to achieve its intended purpose. More specifically, an effective amount means an amount effective to prevent development of or to alleviate the existing symptoms of the subject being treated. Determination of the effective amounts is well within the capability of those skilled in the art, especially in light of the detailed disclosure provided herein. The amount of composition administered will be dependent upon the condition being treated, the subject being treated, on the subjects weight, the severity of the affliction, the manner of administration and the judgment of the personalizing physician.

The ingredients of the dietary supplement of this invention are contained in acceptable excipients and/or carriers for oral consumption. The actual form of the carrier, and thus, the dietary supplement itself, may not be critical. The carrier may be a liquid, gel, gelcap, capsule, powder, solid tablet (coated or non-coated), tea or the like. Suitable excipient and/or carriers include maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, agar, lactose, methylcellulose, povidone, carboxymethylcellulose, corn starch, and the like (including mixtures thereof). The various ingredients and the excipient and/or carrier are mixed and formed into the desired form using conventional techniques. Dose levels/unit can be adjusted to provide the recommended levels of ingredients per day in a reasonable number of units.

The dietary supplement may also contain optional ingredients including, for example, herbs, vitamins, minerals, enhancers, colorants, sweeteners, flavorants, inert ingredients, and the like. Such optional ingredients may be either naturally occurring or concentrated forms. Selection of one or several of these ingredients is a matter of formulation, design, consumer preference and end-user. The amounts of these ingredients added to the dietary supplements of this invention are readily known to the skilled artisan. Guidance to such amounts can be provided by the U.S. RDA doses for children and adults.

### REFERENCES

1. Hadden, J. W. Immunostimulants. Immunol. Today 1993, 14, 275-280.
2. Masihi, K. N. Immunomodulatory agents for prophylaxis and therapy of infections. Int. J. Antimicrob. Agents 2000, 14, 181-191.
3. Van Kampen, K. R. Immunotherapy and cytokines. Semin. Vet. Med. Surg. (Small Anim.) 1997, 12, 186-192.
4. Franz, G. Polysaccharides in pharmacy: Current applications and future concepts. Planta Med. 1989, 55, 493-497.
5. Frank, M. O.; Mandell, G. L. Immunomodulators. In Principles and Practice of Infectious Diseases, chapter 33, 4th th ed.; Mandell, G. L., Bennett, J. E., Dolin, R., Eds.; Churchill Livingstone: New York, 1995; pp 450-458.
6. Wilson, K. Wound healing: the role of macrophages. Nurs. Crit. Care 1997, 2, 291-296.
7. King, G. K.; Yates, K. M.; Greenlee, P. G.; Pierce, K. R.; Ford, C. R.; McAnalley, B. H.; Tizard, I. R. The effect of Acemannan Immunostimulant in combination with surgery and radiation therapy on spontaneous canine and feline fibrosarcomas. J. Am. Animal Hosp. Assoc. 1995, 31, 439-47.
8. Natori, T.; Motoki, K.; Higa, T.; Koezuka, Y. KRN7000 as a new type of antitumor and immunostimulatory drug. In Drugs from the Sea; Fusetani, N., Ed.; Karger: New York, 2000; pp 86-97.
9. Shu, Y. Z. Recent natural products based drug development: A pharmaceutical industry perspective. J. Nat. Prod. 1998, 61, 1053-1071.
10. Ciferri, O, Spirulina, the edible microorganism. Microbiol. Rev. 1983, 47, 551-578.
11. Dantas, D. C.; Kaneno, R.; Queiroz, M. L. The effects of Chlorella vulgaris in the protection of mice infected with Listeria monocytogenes. Role of natural killer cells. Immunopharmacol. Immunotoxicol. 1999, 21, 609-619.
12. Dantas, D. C.; Queiroz, M. L. Effects of Chlorella vulgaris on bone marrow progenitor cells of mice infected with Listeria monocytogenes. Int. J. Immunopharmacol. 1999, 21, 499-508.
13. Qureshi, M. A.; Garlich, J. D.; Kidd, M. T. Dietary Spirulina platensis enhances humoral and cell-mediated immune functions in chickens. Immunopharmacol. Immunotoxicol. 1996, 18, 465-476.
14. Mathew, B.; Sankaranarayanan, R.; Nair, P. P.; Varghese, C.; Somanathan, T.; Amma, B. P.; Amma, N. S.; Nair, M. K. Evaluation of chemoprevention of oral cancer with Spirulina fusiformis. Nutr. Cancer 1995, 24, 197-202.
15. Jensen, G. S.; Ginsberg, D. I.; Huerta, P.; Citton, M.; Drapeau, C. Consumption of Aphanizomenon flos-aquae has rapid effects on the circulation and function of immune cells in humans. JANA 2000, 2, 50-58.
16. Umezawa, I.; Komiyama, K. Acidic polysaccharide CH-1 isolated from Chlorella pyrenoidosa and the use thereof. U.S. Pat. No. 4,533,548, 1985.
17. Watanabe, S.; Seto, A. Ingredient effective for activating immunity obtained from Chlorella minutissima. U.S. Pat. No. 4,831,020, 1989.
18. Watanabe, S.; Fujita, T. Immunopotentiating agent having anti-tumor activity U.S. Pat. No. 4,786,496, 1988.
19. Shinpo, K. Anticancer agent. U.S. Pat. No. 4,822,612, 1989.
20. Noda, K.; Ohno, N.; Tanaka, K.; Okuda, M.; Yadomae, T.; Nomoto, K.; Shoyama, Y. A new type of biological response modifier from Chlorella vulgaris which needs protein moiety to show an antitumor activity. Phytother Res. 1998, 12, 309-319.
21. Tanaka, T.; Yamada, A.; Noda, K.; Hasegawa, T.; Okuda, M.; Shoyama, Y.; Nomoto, K. A novel glycoprotein obtained from Chlorella vulgaris strain CK22 shows antimetastatic immunopotentiation. Cancer Immunol. Immunother. 1998, 45, 313-320.
22. Noda, K.; Ohno, N.; Tanaka, K.; Kamiya, N.; Okuda, M.; Yadomae, T.; Nomoto, K.; Shoyama, Y. A water-soluble antitumor glycoprotein from Chlorella vulgaris. Planta Med. 1996, 62, 423-426.
23. Matsueda, S.; Shinpo, K.; Tanaka, K.; Abe, K.; Karasawa, H. Studies on anti-tumor active glycoprotein from Chlorella vulgaris. II. Sci. Rep. Hirosaki Univ. 1983, 30, 127-131.
24. Morimoto, A.; Nagatsu, A.; Murakami, N.; Sakakibara, J.; Tokuda, H.; Nishino, H.; Iwashima, A. Anti-tumor-promoting glyceroglycolipids from the green alga, Chlorella vulgaris. Phytochemistry 1995, 40, 1433-1437.
25. Mishima, T.; Murata, J.; Toyoshima, M.; Fujii, H.; Nakajima, M.; Hayashi, T.; Kato, T.; Saiki, I. Inhibition of tumor invasion and metastasis by calcium spirulan (Ca-SP), a novel sulfated polysaccharide derived from a blue-green alga, Spirulina platensis. Clin. Exp. Metastasis 1998, 16, 541-550.
26. Lee, J. B.; Hayashi, T.; Hayashi, K.; Sankawa, U.; Maeda, M.; Nemoto, T.; Nakanishi, H. Further purification and structural analysis of calcium spirulan from Spirulina platensis. J. Nat. Prod. 1998, 61, 1101-1104.
27. Hayashi, T.; Hayashi, K.; Kojima, I. Antiviral polysaccharide. U.S. Pat. No. 5,585,365, 1996.
28. Wang, H.; Zeng, H. P.; Yang, S. Z. Isolation, purification and some properties of the water-soluble polysaccharides from Spirulina platensis. Jingxi Huagong 1999, 16, 26-29.
29. Wu, J.; Zhang, C.; Liu, Y. Isolation, purification and immunological activities of extracellular polysaccharide EP II from Spirulina maxima. Yaowu Shengwu Jishu 1999, 6, 99-102.
30. Zhang, Y.; Li, H.; Gao, J.; Shen, Z.; Lin, W.; Fu, H. New process for separation and purification of polysaccharides from Spirulina platensis. Shipin Yu Fajiao Gongye 1999, 25, 15-18.
31. Pasco, D. S.; Pugh, N. D.; Ross, S. A.; ElSohly, H. N.; ElSohly, M. A. Potent immunostimulants from microalgae. U.S. Pat. No. 7,205,284, 2007.
32. Elgert, K. D.; Alleva, D. G.; Mullins, D. W. Tumor-induced immune dysfunction: the macrophage connection. J. Leukoc. Biol. 1998, 64, 275-290.
33. Morrissette, N.; Gold, E.; Aderem, A. The macrophage: a cell for all seasons. Trends Cell. Biol. 1999, 9, 199-201.
34. Gordon, S. The role of the macrophage in immune regulation. Res. Immunol. 1998, 149, 685-688.
35. Adams, D. O.; Hamilton, T. A. Molecular basis of macrophage activation: diversity and its origins. In The Natural Immune System: The Macrophage; Lewis, C. E., McGee, J. O'D., Eds.; Oxford University Press Inc.: New York, 1992; pp 75-114.
36. May, M. J.; Ghosh, S. Signal transduction through NF-kappa B. Immunol Today 1998, 19, 80-88.
37. Baeuerle, P. A.; Henkel, T. Function and activation of NF-kappa B in the immune system. Annu. Rev. Immunol. 1994, 12, 141-179.
38. Chang, C. C.; Zhang, J.; Lombardi, L.; Neri, A.; Dalla-Favera, R. Mechanism of expression and role in transcriptional control of the proto-oncogene NFKB-2/LYT-10. Oncogene, 1994, 9, 923-933.
39. Braun, V.; Wu, H. C. Lipoproteins, structure, function, biosynthesis and model for protein export. New Compr. Biochem. 1994, 27, 319-341.
40. Identification of S-(2,3-dihydroxypropyl)cystein in a macrophage-activating lipopeptide from Mycoplasma fermentans. Biochemistry, 1996, 35, 7781-7786.
41. Hashimoto M, Tawaratsumida K, Kariya H, Aoyama K, Tamura T, Suda Y. Lipoprotein is a predominant Toll-like receptor 2 ligand in Staphylococcus aureus cell wall components. Int Immunol. 2006, 18, 355-362.

1.-25. (canceled)

26. An immunostimulatory composition that comprises a lipoprotein preparation obtained as an extract of one of the following microalgae or any combination thereof: Spirulina species, Haematococcus pluvialis, Chlorella species, or Aphanizomenon flos-aquae,
wherein the lipoprotein preparation is obtained by extraction of the microalgae with a solvent comprising a detergent, a surfactant, an emulsifier or any combination thereof
or
wherein the microalgae is extracted with a first solvent comprising water, alcohol or a non-polar solvent
and
the lipoprotein preparation is obtained by further extraction of the microalgae with a second solvent comprising a detergent, a surfactant, an emulsifier or any combination thereof.

27. An immunostimulatory composition that comprises a lipoprotein preparation obtained as an extract of Haematococcus pluvialis.

28. The immunostimulatory composition of claim 26, wherein the lipoprotein preparation comprises an extract of Spirulina species, Chlorella species, Haematococcus pluvialis, or Aphanizomenon flos-aquae.

29. The immunostimulatory composition of claim 27, wherein the lipoprotein preparation comprises an extract of Haematococcus pluvialis.

30. The immunostimulatory composition of claim 27, wherein the lipoprotein preparation is obtained by extraction of the microalgae with a solvent comprising aqueous alcohol, detergent, a surfactant, an emulsifier or any combination thereof.

31. The immunostimulatory composition of claim 26, wherein the microalgae is Spirulina platensis.

32. The immunostimulatory composition of claim 26, wherein the microalgae is Chlorella pyrenoidosa.

33. The immunostimulatory composition of claim 27, wherein the microalgae is extracted with a first solvent and the lipoprotein preparation comprises an extract of the extracted microalgae with a second solvent.

34. The immunostimulatory composition of claim 33, wherein the first solvent comprises water, alcohol, or a non-polar solvent.

35. The immunostimulatory composition of claim 33, wherein the second solvent comprises a solvent containing aqueous alcohol, a detergent, a surfactant, an emulsifier or any combination thereof.

36. The immunostimulatory composition of claim 26, wherein the composition manifests immunostimulation by monocyte and/or macrophage activation.

37. The immunostimulatory composition of claim 27, wherein the composition manifests immunostimulation by monocyte and/or macrophage activation

38. A method of treating a subject requiring immune mediation comprising administering to said subject the immunostimulatory composition of claim 26.

39. A method of treating a subject requiring immune mediation comprising administering to said subject the immunostimulatory composition of claim 27.

40. An immunostimulatory agent, comprising: an immunostimulatory effective amount of the immunostimulatory composition of claim 26 and an acceptable carrier or excipient.

41. An immunostimulatory agent, comprising: an immunostimulatory effective amount of the immunostimulatory composition of claim 27 and an acceptable carrier or excipient.

42. An adjuvant agent, comprising: an immunostimulatory effective amount of the immunostimulatory composition of claim 26 and an acceptable carrier or excipient.

43. An adjuvant agent, comprising: an immunostimulatory effective amount of the immunostimulatory composition of claim 27 and an acceptable carrier or excipient.

44. An immunostimulatory composition comprising microalgae-derived lipoproteins, wherein the microalgae comprises one of the following or any combination thereof: Spirulina species, Chlorella species, Haematococcus pluvialis, or Aphanizomenon flos-aquae.

45. The immunostimulatory composition of claim 44 in which said lipoproteins contain a diacyl glycerol moiety attached via a thioether to a N-terminal cysteine of said lipoproteins.

46. A method for preparing a bioactivity standardized product containing an effective amount of immunostimulatory lipoproteins, comprising:
(a) providing a microalgae material selected from the group of Spirulina species, Chlorella species, Haematococcus pluvialis or Aphanizomenon flos-aquae.
(b) extracting the microalgae material with a solvent to produce an extract.
(c) optionally purifying the extract.
(d) testing in vitro the extract for activation of immune cells.
(e) comparing the activity of the extract to a standard preparation immunostimulatory value to determine a standardized activity value of the product.

47. A method for preparing a chemically standardized product containing an effective amount of immunostimulatory lipoproteins, comprising:
(a) providing a microalgae material selected from the group of Spirulina species, Chlorella species, Haematococcus pluvialis or Aphanizomenon flos-aquae.
(b) extracting the microalgae material with a solvent to produce an extract.
(c) optionally purifying the extract.
(d) testing the extract for a chemical marker specific to immunoactive lipoproteins, wherein said chemical marker is 2,3-dihydroxypropyl cysteine.
(e) comparing the amount of chemical marker in the extract to the amount of chemical marker in a standard preparation to determine a standardized activity value of the product.

48. The method of claim 46, wherein the standardized product is whole microalgae or microalgae spent material selected from one of the following or any combination thereof: Spirulina species, Chlorella species, Haematococcus pluvialis or Aphanizomenon flos-aquae.

49. The method of claim 47 wherein the standardized product is whole microalgae or microalgae spent material selected from one of the following or any combination thereof: Spirulina species, Chlorella species, Haematococcus pluvialis or Aphanizomenon flos-aquae.

50. The method of claim 46, wherein the standardized product is an extract of one of the following microalgae or any combination thereof: Spirulina species, Chlorella species, Haematococcus pluvialis or Aphanizomenon flos-aquae.

51. The method of claim 47, wherein the standardized product is an extract of one of the following microalgae or any combination thereof: Spirulina species, Chlorella species, Haematococcus pluvialis or Aphanizomenon flos-aquae.

52. A method of providing a dietary supplement in a subject requiring enhanced immune system support to enhance said subject's immune system; comprising providing an effective immune cell activating amount of the composition of claim 26, and administering said composition to said subject.

53. A method of providing a dietary supplement in a subject requiring enhanced immune system support to enhance said subject's immune system; comprising providing an effective immune cell activating amount of the composition of claim 27, and administering said composition to said subject.

54. The method of claim 52 in which said composition is obtained after a first extraction with a solvent that has removed materials other than lipoproteins.

55. The method of claim 53 in which said composition is obtained after a first extraction with a solvent that has removed materials other than lipoproteins.

56. A method for treating, preventing, or ameliorating a condition or disease in a subject requiring enhanced immune system support comprising providing an effective immune cell activating amount of a microalgae spent material and administering spent material to said subject.

57. A method of providing a dietary supplement to a subject requiring enhanced immune system support to enhance said subject's immune system; comprising providing an effective immune cell activating amount of a microalgae spent material and administering spent material to said subject.
